(19) United States
(12) Patent Application Publication
(10) Pub. No.: US 2009/0061416 A1
(43) Pub. Date: Mar. 5, 2009

(54) Surfaces and methods for biosensor cellular assays

(76) Inventors: Ye Fang, Painted Post, NY (US); Ann M. Ferrie, Painted Post, NY (US); Elizabeth Tran, Painted Post, NY (US); Jun Xi, Ardmore, PA (US)

Correspondence Address: CORNING INCORPORATED, SP-TI-3-1, CORNING, NY, 14831, US

(21) Appl. No.: 12/072,599

(22) Filed: Feb. 27, 2008

Related U.S. Application Data
(60) Provisional application No. 60/904,129, filed on Feb. 28, 2007.

(51) Int. Cl. C12Q 1/70; C12M 1/00; C12N 11/00; G01N 33/569; G01N 33/53

(52) U.S. Cl. 435/5; 435/288.7; 435/174; 435/7.2; 435/7.32

(57) ABSTRACT

Disclosed is an apparatus for measuring ligand-induced cell activity as defined herein, the apparatus including: an optical biosensor having a contact surface including a compatibilizer zone, an optional surface modifier zone, and a live cell zone. The disclosure also provides a method of making the apparatus and methods for measuring ligand-induced live cell activity with the apparatus.

### CLAIMING BENEFIT OF PRIOR FILED U.S. APPLICATION

This application claims the benefit of U.S. Provisional Application Ser. No. 60/904,129, filed on Feb. 28, 2007. The content of this document and the entire disclosure of publications, patents, and patent documents mentioned herein are incorporated by reference.

### BACKGROUND

The disclosure relates to optical biosensors, and more specifically to resonant waveguide grating (RWG) biosensors and methods for cellular assays such as live cell sensing. For a related disclosure see, for example, commonly-owned, copending PCT application, entitled “Label-Free Biosensors and Cells,” Y. Fang et al., PCT App. No. PCT/US2006/013539 (Pub. No. WO 2006/108183), published Dec. 10, 2006.

The ability to examine living cells in native or physiological-relevant contexts is desirable to better understand the biological functions of cellular targets, and to more accurately assess the success of drug discovery and development. Although more complex and less specific than biochemical assays, cell-based assays that can monitor the activity and health of living cells have gained popularity, for example, in drug discovery and development because cell-based assays can provide a significant benefit of extracting functional information that would otherwise be lost with biochemical assays. Cell-based assays can also facilitate the measurement of, for example, mode-of-action, pathway activation, toxicity, phenotypic responses, and like responses of cells mediated by exogenous stimuli. Conventional cell-based assays can measure a specific cellular event, for example, second-messenger generation, translocation of a particular target tagged with a fluorescent label, expression of a reporter gene, alteration of a particular phenotype, and like events. However, compared to biochemical assays wherein isolated biomacromolecules such as protein(s) are assayed directly, current cell-based assays require more manipulations, for example, over expression of targets with and without a readout tag (e.g., fluorescent molecules such as green fluorescent protein); and such manipulations could pose significant issues to the cellular physiology of the targets of interest. For compound library screening, which is widely used for drug discovery and development or biomarker identification, the number of compounds that could interfere with the detection methodologies may be significant, thus leading to great number of false positives or negatives. For example, colored compounds could interfere with the fluorescence-based detection methods. Thus, a cell-based assay that can provide, for example, a non-invasive and manipulation-free detection of cellular activity with high sensitivity is highly desirable.

However, challenges remain for optical biosensors and their use. For example, the high sensitivity of optical biosensors to slight changes in refractive index of the medium, an enabling strength, can pose a complication such as in drug discovery assays where buffered solutions and samples may need to be calibrated for polar solvent content, such as DMSO. See for example Hitt, E., “Label Free not without Problems” in Drug Discovery & Development (www.dddmag.com, Jan. 21, 2005; http://cmliris.harvard.edu/news/2004/DrugDiscDev_Sep04.pdf. Another challenge is that label-free or label-independent-detection (LID) methods using optical biosensors while rapid, such as in high throughput screening (HTS) applications, can have reproducibility issues.

There remains a need for a robust optical biosensor apparatus and methods for measuring ligand-induced live cell activity which overcome the aforementioned challenges and related challenges mentioned herein of existing apparatus and methods.

### SUMMARY

In general terms, the claimed invention relates to optical biosensors, and more specifically to resonant waveguide grating (RWG) biosensors and methods for cellular assays such as live cell sensing. The present disclosure provides an apparatus for measuring ligand-induced cell activity as defined herein, the apparatus including: an optical biosensor having a contact surface including a compatibilizer zone, an optional a surface modifier zone, and a cell zone. The disclosure also provides a method of making the apparatus and methods for measuring ligand-induced cell activity with the apparatus.

The present disclosure provides surfaces and methods for cell-based assays using biosensors, for example, evanescent wave-based optical biosensors including surface plasmon resonance (SPR) and RWG biosensors. The disclosed surfaces provide for appropriate cell growth and cell attachment, and enable assays for ligand-mediated cellular activities. In embodiments, the present disclosure provides surfaces having a thin layer of inorganic materials for a wide range of adherent cells, such as Chinese hamster ovary (CHO) cells and other mammalian cell lines. In embodiments, the present disclosure provides surfaces having cell anchoring materials or compatibilizers for weakly adherent cells such as human embryonic kidney (HEK) cells and engineered HEK cells, as well as suspension cells such as T cells or killer cells. In embodiments, the disclosure also provides a method to study ligand-induced intracellular activity of cell suspensions, whose activities typically cannot be detected by conventional optical biosensors since they generally only permit the measurements of ligand-induced dynamic redistribution of cellular contents within the bottom thin portion of adherent cell layer, but not cell suspensions. In embodiments, the present disclosure provides a biosensor microtiter plate device having many different types of surfaces and the use of such device for rapid screening and identification of an optimal surface that enables robust biosensor cell-based assays for a native cell line or an engineered cell line having, e.g., an over-expressed target.

The assay methods of the disclosure are well suited, for example, for use with weakly adherent cells and suspension cells that may have ligand induced activity that cannot otherwise be detected using biosensors with a limited sensing volume.

### BRIEF DESCRIPTION OF THE DRAWINGS

FIGS. 1A and 1B show cross-sectional configurations of two examples of RWG biosensors used for living cell sensing, in embodiments of the present disclosure.

FIGS. 2A and 2B shows the results of adenosine triphosphate (ATP)-induced optical signals obtained from CHO-KCNQ cells on the respective configurations of FIGS. 1A and 1B, in embodiments of the disclosure.

FIG. 3 is a schematic that illustrates clusters or islands of a cell anchoring material or compatibilizer on the surface of a RWG biosensor, wherein the clusters can be anchor points for cell attachment and growth, in embodiments of the disclosure.

FIGS. 4A and 4B show two example configurations of surface modified RWG biosensors used for living cell sensing and which biosensors have differential sensitivities to stimulus-induced change in local mass or mass density, in embodiments of the disclosure.

FIG. 5 compares the ATP-induced dynamic mass redistribution signals obtained from HEK293 cells that were cultured onto three different surfaces having different gelatin coating densities, in embodiments of the disclosure.

FIGS. 6A and 6B show the reproducibility and robustness of a cell assay using HEK293 cells cultured on a low-density gelatin-coated 384-well biosensor microplate (Epic™ cell plates, Corning Inc.), in embodiments of the disclosure.

FIG. 7 shows a standard activity assay plot of ATP-induced optical signals for HEK293 cells cultured on RWG surfaces having different concentrations of surface bound tripeptide, in embodiments of the disclosure.

FIG. 8A shows a plot of optical signals obtained from Jurkat cells before and after stimulation with a cell permeable peptide (pseudo-RACK1), on which the suspension cells are anchored onto biosensors having different surface preparations and properties, in embodiments of the disclosure.

FIG. 8B shows a schematic of a possible mechanism for a ligand-induced cellular event of a Jurkat cell and its sensing result using a biosensor, in embodiments of the disclosure.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In embodiments the disclosure provides an apparatus for measuring ligand-induced cell activity, the apparatus including: an optical biosensor having a contact surface including: a compatibilizer zone having a compatibilizer in contact with the surface of the biosensor; and a cell zone having at least one cell associated with at least one compatibilizer, the compatibilizer can include, e.g., at least one of: an isolated compatibilizer, an island of two or more compatibilizers, a discontinuous film comprised of a plurality of compatibilizers, or combinations thereof. In embodiments the disclosure also provides a method of making the apparatus and methods for measuring ligand-induced cell activity with the apparatus.

### DEFINITIONS

“Assay,” “assaying” or like terms refers to an analysis to determine, for example, the presence, absence, quantity, extent, kinetics, dynamics, or type of a cell's optical response upon stimulation with an exogenous stimuli, such as a ligand candidate compound.

“Attach,” “attachment,” “adhere,” “adhered,” or like terms generally refer to immobilizing or fixing, for example, a surface modifier substance, a compatibilizer, a cell, a ligand candidate compound, and like entities of the disclosure, to a surface, such as by physical absorption, chemical bonding, and like processes, or combinations thereof. Particularly, “cell attachment”, “cell adhesion”, or like terms refer to the interacting or binding of cells to a surface, such as by culturing, or interacting with cell anchoring materials, compatibilizer, or both.

“Adherent cells” refers to a cell or a cell line, such as a prokaryotic or eukaryotic cell, that remains associated with, immobilized on, or in certain contact with the outer surface of a substrate during cell culture. Such type of cells after culturing can withstand or survive washing and medium exchanging process, a process that is prerequisite to many cell-based assays. “Weakly adherent cells” refers to a cell or a cell line, such as a prokaryotic or eukaryotic cell, that weakly interacts or associates or contacts with the surface of a substrate during cell culture. However, these types of cells, for example, HEK cells, tend to dissociate easily from the surface of a substrate by physically disturbing approaches such as washing or medium exchange. “Suspension cells” refers to a cell or a cell line that is preferably cultured in a medium wherein the cells do not attach or adhere to the surface of a substrate during the culture. “Cell culture” or “cell culturing” refers to the process by which either prokaryotic or eukaryotic cells are grown under controlled conditions. “Cell culture” not only refers to the culturing of cells derived from multicellular eukaryotes, especially animal cells, but also the culturing of complex tissues and organs.

“Compatibilizer,” “cell anchoring material,” or like terms refer to a molecule or material, naturally occurring or synthetic, which can be applied to a biosensor surface to render it more receptive or interactive with a subsequently contacted or cultured cell so as to enhance immobilization of the cell to the biosensor. A compatibilizer can directly interact with a cell. Such compatibilizer can include, for example, polypeptides, antigens, polyclonal antibodies, monoclonal antibodies, single chain antibodies (scFv), F(ab) fragments, F(ab′)₂ fragments, Fv fragments, peptides, proteins, naturally occurring- and denatured cell adhesion polypeptides, polymers, reactive molecules, and like materials or molecular entity, which can specifically bind to or interact with at least one of any of a cell's surface molecules. In embodiments, a compatibilizer can indirectly interact with a cell wherein the compatibilizer leads to the releasing of cell adhesion molecules and subsequent formation of extracellular matrix (ECM). Such a compatibilizer can include, for example, organic compounds, such as small molecules and polymers, for example an aminosilane, a polyalkylene glycol, or mixtures thereof, and inorganic materials, such as water insoluble metal oxide or mixed metal oxides, and inorganic polymers such as silica. For additional definitions, descriptions, and methods of silica (silicon dioxide, SiO₂) materials and related metal oxide materials as used herein, see for example, R. K. Iler, The Chemistry of Silica, Wiley-Interscience, 1979.

“Compatibilization,” “compatibilized,” or like terms refer to the act or result of applying a compatibilizer to a biosensor surface to render the surface compatible or receptive to cell attachment.

“Surface modifier” or like term refers to a molecule or material, naturally occurring or synthetic, which can be applied to a biosensor surface to render it more receptive to or interactive with a subsequently applied compatibilizer so as to enhance immobilization of the compatibilizer to the biosensor surface.

“Cell” or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including synthetic cell constructs, cell model systems, and like artificial cellular systems.

“Ligand candidate compound,” “ligand candidate,” “candidate,” or like terms refer to a molecule or material, naturally occurring or synthetic, which is of interest for its potential to interact with a cell attached to the biosensor. A ligand candidate can include, for example, a chemical compound, a biological molecule, a peptide, a protein, a biological sample, a drug candidate small molecule, a drug candidate biologic molecule, a drug candidate small molecule-biologic conjugate, and like materials or molecular entity, or combinations thereof, which can specifically bind to or interact with at least one of a cellular target such as a protein, DNA, RNA, an ion, a lipid or like structure or component of a living cell.

“Biosensor” or like term refers to a device for the detection of an analyte that combines a biological component with a physicochemical detector component. The biosensor typically consists of three parts: a biological component or element (such as tissue, microorganisms and cells), a detector element (works in a physicochemical way such as optical, piezoelectric, electrochemical, thermometric, or magnetic), and a transducer associated with both components. The biological component or element can be, for example, a living cell. In embodiments, an optical biosensor can comprise an optical transducer for converting a molecular recognition or molecular stimulation event in a living cell into a quantifiable signal.

Abbreviations, which are well known to one of ordinary skill in the art, may be used (e.g., “h” for hour or hours, “g” or “gm” for gram(s), “mL” for milliliters, and “rt” for room temperature, “nm” for nanometers, and like abbreviations).

“Include,” “includes,” or like terms means including but not limited to.

“About” modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

“Consisting essentially of” in embodiments refers, for example, to a surface composition, a method of making or using a surface composition, formulation, or composition on the surface of the biosensor, and articles, devices, or apparatus of the disclosure, and can include the components or steps listed in the claim, plus other components or steps that do not materially affect the basic and novel properties of the compositions, articles, apparatus, and methods making and use of the disclosure, such as particular reactants, particular additives or ingredients, a particular pre-treatment or blocking agent, a particular cell or cell line, a particular surface modifier, a particular compatibilizer, a particular cell or cell line, a particular ligand candidate, or like structure, material, or process variable selected. Items that may materially affect the basic properties of the components or steps of the disclosure or may impart undesirable characteristics to the present disclosure include, for example, decreased affinity of the cell for the biosensor surface, decreased affinity of the ligand candidate for a cell, anomalous or contrary cell activity in response to a ligand candidate, and like characteristics.

The indefinite article “a” or “an” and its corresponding definite article “the” as used herein means at least one, or one or more, unless specified otherwise.

Specific and preferred values disclosed for reactants, ingredients, additives, and ranges thereof, are for illustration only; they do not exclude other defined values or other values within defined ranges. The compositions, apparatus, and methods of the disclosure include those having any value or any combination of the values, specific values, more specific values, and preferred values described herein.

The specific compositions or ingredients used in the preparation of the compositions of the disclosure, and like compositions, can include suitable salt or salts thereof or as illustrated herein. The starting materials employed in the methods described herein are commercially available, have been reported in the literature, or can be prepared from readily available starting materials using procedures known in the field. In embodiment, relative proportions of the reactants can be varied depending on properties desired in the resulting biosensor surface composition, such as porosity, density, surface area coverage, and like properties of the surface modifier, the compatibilizer, the cell, and combinations thereof.

In embodiments the disclosure provides an apparatus for measuring ligand-induced cell activity, the apparatus can comprise, for example: an optical biosensor having a contact surface comprising: a compatibilizer zone having a compatibilizer in contact with the surface of the biosensor; and a cell zone having at least one cell associated, for example, attached to or immobilized with at least one compatibilizer. In embodiments the compatibilizer zone can comprise a compatibilizer directly or indirectly connected to the surface of the biosensor. If directly connected there is no intervening material or substance. If indirectly connected there is an intervening material or substance, such as a surface modifier. In embodiments the compatibilizer can be a continuous but non-uniform film or layer, such as a having complete biosensor coverage but having a non-uniform film thickness. In embodiments the compatibilizer can be a discontinuous film or layer, such as a having incomplete biosensor coverage, for example, having coverage gaps that can be minor or minimal, intermediate, or major or considerable, and having a uniform or non-uniform film thickness. In embodiments the compatibilizer can be a discontinuous film or layer having intermediate or considerable biosensor surface area coverage gaps and can comprise, for example, at least one of an isolated or single compatibilizer, a patch or an island of two or more compatibilizers, a thin discontinuous film comprised of a plurality of compatibilizers, or combinations thereof.

In embodiments the compatibilizer zone can comprise, for example, a surface modifier as continuous or discontinuous film or layer on the biosensor surface having a uniform or non-uniform film thickness and a compatibilizer. In embodiments the compatibilizer zone can comprise, for example, a surface modifier as continuous film or layer on or in contact with the biosensor having a uniform or non-uniform film thickness, and a discontinuous film or layer of a compatibilizer associated with some or much of the surface modifier but not all of the surface modifier. In embodiments the compatibilizer is not a uniform, continuous film.

In embodiments the cell can comprise, for example, at least one of a surface adherent cell, a weakly adherent cell, a cell suspension, or combinations thereof.

In embodiments the compatibilizer can comprise, for example, a gelatin, a peptide, an antibody, a nanoparticle, and like entities, or combinations thereof on the biosensor at a low density or concentration, for example, having a surface coverage on the biosensor surface of from about 0.01% to about 10%, of from about 0.1% to about 10%, of from about 0.1% to about 5%, of from about 0.1% to about 2%, of from about 0.1% to about 1 weight %, and like surface coverage. The compatibilizer can be applied, for example, to a bare biosensor surface or to a surface modifier treated biosensor surface, for example, at an effective concentration such as from about 1 to about 1,000 micromolar, from about 1 to about 500 micromolar, from about 1 to about 250 micromolar, or like effective concentrations. Subsequent washing, stripping, or like treatments, can reduce the surface concentration of the compatibilizer to, for example, the abovementioned surface coverages, or like coverages.

In embodiments the compatibilizer zone can comprise, for example, the region occupied by a compatibilizer and situated between the biosensor's surface and a cell. In embodiments the compatibilizer zone can further include, for example, a surface modifier zone which can comprise, for example, the region occupied by a surface modifier situated between the biosensor's surface and the compatibilizer. In embodiments the surface modifier can comprise, for example, at least one nanoparticulate of: a metal oxide, a mixed metal oxide, a surface treated metal oxide, a surface treated mixed metal oxide, or combinations thereof, and having a layer thickness average particle diameter, for example, of from about 1 to about 1,000 nanometers, of from about 1 to about 100 nanometers, of from about 1 to about 50 nanometers, of from about 1 to about 15 nanometers, and like dimensions and ranges. In embodiments the nanoparticulate can be, for example, a nanoparticulate silica, and can be present on the surface of the biosensor, for example, in a surface coverage amount of from about 0.01% to about 10%, or like coverages. In embodiments the surface modifier or compatibilizer can be gelatin which can be, for example, a nanoparticulate, or a discontinuous film or layer, and can be present on the surface of the biosensor, for example, in a surface coverage amount of from about 0.01% to about 10%, or like coverages

In embodiments the disclosure provides a method of making the aforementioned apparatus. In embodiments the method comprises, for example, decorating a surface of an optical or like biosensor with a compatibilizer to form a compatibilized biosensor contact surface; and attaching a cell to the compatibilizer-decorated biosensor surface. The decorating results in a biosensor surface having, for example, from about 10 to about 95 percent compatibilizer coverage based on the available biosensor contact surface area. Attaching a cell to the compatibilizer decorated biosensor surface results in a sensing surface having, for example, from about 10 to about 100 percent of available compatibilizer surface or compatibilizer sites covered by associated cells. The decorating, in embodiments, can further comprise, for example, contacting the biosensor surface with a surface modifier prior to contacting the biosensor surface with a compatibilizer.

In embodiments the cell attaching can be accomplished by, for example, contacting the compatibilizer-decorated biosensor surface with a cell as illustrated and demonstrated herein.

In embodiments the method of making the apparatus can further comprise, for example, treating the compatibilized biosensor surface with a blocking agent prior to contacting the compatibilized biosensor surface with a cell suspension. A suitable blocking agent can be, for example, an hydrophilic amine or thiol compound such as ethanolamine, diethanolamine, thioethanol, or like compounds, and combinations thereof, as applied to the surface, for example as an aqueous solution at from about 0.01 to about 10 wt %, from about 0.1 to about 5 wt %, from about 0.1 to about 1 wt %, or like concentrations.

In embodiments the disclosure provides a method of measuring ligand-induced cell activity, the method comprising: contacting the above described surface compatibilized optical biosensor with a ligand candidate; and measuring the cell's optical response to the ligand contact with, for example, a suitable optical detection system.

In embodiments the ligand candidate can be, for example, at least one of a drug candidate small molecule, a drug candidate biologic molecule, a drug candidate small molecule-biologic conjugate, a virus, a bacterium, and like ligand candidate entities as disclosed and illustrated herein, or combinations thereof. The ligand candidate can be selected by for example, laboratory screening or similar methods, to have no or low affinity with, for example, the uncoated biosensor surface; a surface modifier treated biosensor surface; a compatibilizer treated biosensor surface; or a surface modifier and compatibilizer treated biosensor surface. Conversely, the surface modifier, compatibilizer, or both, can be selected by for example, laboratory screening or similar methods, to have no or low affinity with the ligand.

In embodiments measuring the cell's optical response to the ligand contact can comprise, for example, detecting and determining the difference between the refractive index of the incident and reflected light by methods understood by one skilled in the art. In embodiments measuring the cell's optical response to the ligand contact can further comprise, for example, correlating the DMR signals to the cell's activity.

In embodiments the disclosure provides a method to assay ligand-induced cell activity, the method comprising, for example: contacting a medium having at least one cell therein with a surface of an optical biosensor, the biosensor surface having a compatibilizer attached to the biosensor surface and the compatibilizer having a functional group that can interact with a cell surface molecule; incubating the medium with the surface of the optical biosensor until a cell attaches to the biosensor surface; contacting the biosensor having an attached cell with a ligand candidate or like compound; optionally removing unattached cells and optionally removing medium; and monitoring the cell response to the ligand contact with a detection system. In embodiments the biosensor surface having a compatibilizer attached to the biosensor surface can optionally include a surface modifier that is, situated between the compatibilizer and the biosensor surface.

In embodiments the incubating can be followed by removing or separating any unattached cells and optionally medium from the treated biosensor surface. In embodiments the biosensor surface having a compatibilizer attached to the biosensor surface can include, for example, a surface modifier situated between the compatibilizer and the biosensor surface.

In embodiments the disclosure provides a method for making a cell-based biosensor, the method comprising, for example,

contacting a biosensor having a receptive surface with a compatibilizer formulation, such as solution or suspension of a suitable compatibilizer, the compatibilizer having a concentration of, for example, from about 1 to about 1,000 micromolar, from about 1 to about 500 micromolar, from about 10 to about 250 micromolar, from about 10 to about 100 micromolar, or like concentrations and ranges, to form a biosensor having a surface coated with the compatibilizer;

washing, and optionally drying, the resulting compatibilizer coated biosensor surface with a suitable liquid to remove unbound compatibilizer to form a compatibilized biosensor having a surface decorated with compatibilizer; and

contacting the compatibilized biosensor surface with a cell suspension to form a compatibilized biosensor having one or more cells of the cell suspension attached to the compatibilized biosensor surface. The method can further comprise, e.g., treating the compatibilized biosensor surface with a blocking agent or like treatments before accomplishing cell interactions.

In embodiments the method can further comprise, e.g., contacting, and thereafter optionally washing, the compatibilized biosensor surface having a cell attached or immobilized thereon with a ligand candidate compound to form a compatibilized biosensor having at least one or more ligand candidate bound to or associated with at least one cell associated with the compatibilized biosensor surface.

In embodiments where the aforementioned method of contacting the compatibilized biosensor surface having an attached cell with a ligand candidate does not bind to or associate with at least one attached cell a useful negative result is obtained indicating, for example, that the concentration, the rate of reaction, the affinity, or like considerations, of the ligand candidate for a receptor or like surface molecule of the biosensor bound cell is insufficient under the specified assay conditions. Thus, the method can be used as a sensitive discriminator of, for example, a combinatorial library of ligand candidates, such as screening lead pharmaceutical compounds or biologic therapeutic candidates.

In embodiments, the disclosure also provides a method to assay ligand-induced cell activity, the method comprising: incubating a medium having at least one cell therein with a contact surface of an optical biosensor until a cell attaches to the biosensor surface, the biosensor surface having a compatibilizer attached-to but incompletely covering the biosensor surface, the compatibilizer having a functional group that can interact with a cell surface molecule; contacting the biosensor having an attached cell with a ligand candidate; and monitoring the cell response to the ligand contact with a detection system.

In embodiments, the disclosed surface modified biosensors and methods of making and using surface modified biosensors enable application of biosensor cell-based assays to diverse cell types and cell dispositions, such as adherent cells, weakly adherent cells, suspension cells, and like cell dispositions, or combinations thereof. Most evanescent wave-based optical biosensors have a well-defined and characterized penetration depth or sensing volume or detection zone at or near the sensor surface, in which the evanescent wave only extends into the solution or a cell layer with a short distance (typically less than about 200 nm). Because of the short sensing volume of commonly available optical biosensors, including RWG and surface plasmon resonance (SPR), biosensor cell-based assays call for close proximity of the cells to the sensor surface of, for example, several hundreds of nanometers. Additionally, surface attachment and growth of cells can be significant factors in achieving success with a robust biosensor cell-based assay. In embodiments, the biosensor contact surface should be biocompatible with and support the attachment and growth of a wide variety of cell lines. In embodiments, the cells adhered to the biosensor contact surface can withstand manipulations such as washing and reagent dispensing.

In embodiments, the disclosure provides a method to achieve a low-density coating of a biosensor surface with a biologically compatible material, such as gelatin or a compatibilizer. The resulting coated surface allows a wide range of cells, particularly weakly adherent cells, such as HEK cells, to attach to and grow on the coated surface, and enable robust cell assays, for example, detection and resolution of the interaction of the biosensor attached cells with a ligand candidate entity.

In embodiments, the disclosure also provides a biosensor having a coated surface such as an incomplete or discontinuous coat of compatibilizer, surface modifier, or both, that allow a cell or cell suspensions to interact with the coated surface. Additionally, the disclosure provides methods to detect and measure ligand-induced cellular activities of cell suspensions using the biosensor.

In embodiments, the present invention provides a biosensor microtiter plate article or device having at least one of a variety of different surfaces and the use of such device for rapid screening and identification of an optimal surface that enables, for example, robust biosensor cell-based assays, e.g., for a native cell line or an engineered cell line having an over-expressed target. Because of the diversity of cell types of interest, as well as the unexpected impact of an over-expressed target in a known and well-characterized cell line, the attachment, growth, and assays with biosensors of a native and engineered cell line may require different surface chemistries in order to achieve optimal performance. Thus, an article or device having the capability of and adaptability for multiple surface chemistries is desired to screen and identify optimal surfaces for biosensor-based cell assays. Such as device is disclosed and illustrated herein.

### Optical Biosensors for Live Cell Sensing

Historically, optical biosensors were used primarily for routine biomolecular interaction analysis because of their abilities to provide detailed information on the binding affinity and kinetics of a biomolecular interaction. Thus, these devices have often been referred to as affinity-based biosensors. To increase the productivity of drug discovery, drug discovery paradigms have been shifting from a target-directed approach to the systems biology-centered approach in recent years. Such paradigm shift calls for the use of living cell systems for testing and drug screens, thus creating both a need and a potential solution of drug discovery.

Similar to other types of biosensors that use, for example, calorimetric, acoustic, electrochemical, or magnetic transducers, the optical biosensors of the present disclosure comprise optical transducers for converting a molecular recognition event or a ligand-induced cellular response into a quantifiable signal, termed an optical signal. For living cell sensing, a cell system or a biological system containing cells (e.g., live cells, tissue, a tumor, blood or like bodily fluids, bacteria, and like specimens) is contacted with the surface of a biosensor of the disclosure to form, for example, a biofilm, a cell layer, or like decoration of the cell system or the biological system on the compatibilizer decorated biosensor surface. The interaction between a target analyte, that is, a ligand candidate, such as a drug candidate compound, and the layer of the cell system or the biological system on the biosensor surface produces a change in the optical content of the reflected light. Such changes can be detected by the transducer and used to determine the target molecule-induced alterations of the layer of the cell system or like biological system associated with the surface of the biosensor.

During the past several decades, a variety of optical biosensors have been developed, including, for example, surface plasmon resonance (SPR), resonant waveguide grating (RWG), and resonant mirrors, and like optical biosensors. Almost of all these optical biosensors exploit evanescent waves to characterize molecular interactions or alterations of a layer of the cell system or the biological system at or near the sensor surface. The evanescent-wave is an electromagnetic field, created by the total internal reflection of light at a solution-surface interface, which typically extends a short distance, for example several hundreds of nanometers, into the solution having a characteristic depth, termed the penetration depth or the sensing volume or the detection zone. In embodiments the surface modifier zone, the compatibilizer zone, and at least a portion of the associated cell or cell zone, or like biological system resides in the detection zone. The present disclosure is applicable to these evanescent wave-based optical biosensors for whole cell sensing.

Specifically, an RWG biosensor utilizes the resonant coupling of light into a waveguide by means of a diffraction grating. There are many types of detection schemes, for example, wavelength and angular interrogation systems.

In a wavelength interrogation system, polarized light, covering a range of incident wavelengths, is used to directly illuminate the waveguide; light at specific wavelengths is coupled into and propagates along the waveguide. The resonance wavelength at which a maximum in-coupling efficiency is achieved is a function of the local refractive index at or near the sensor surface. When a target molecule in a sample binds to a cellular target and subsequently triggers a dynamic relocation or redistribution of cellular contents within the bottom portion of the layer of the cell system or the biological system (i.e., within the detection zone or sensing volume of the optical biosensor), and is accompanied by a shift in the resonance wavelength. Although not wanting to be limited by theory the dynamic relocation or redistribution of cellular content could be attributable to, e.g., the dynamic relocation of any cellular targets, the change in the morphology (such as cell rounding or flattening, or cytoskeletal remodeling) of the cell system induced by the stimulation of the cell system with the ligand, or both.

An example of a commercial instrument embodying the resonance wavelength method is the Corning® Epic™ system (www.corning.com/lifesciences), which includes an RWG detector having, for example, a temperature-controlled environment and a liquid handling system. The detector system includes integrated fiber optics to measure the ligand-induced wavelength shift of the reflected light. A broadband light source, generated through a fiber optic and a collimating lens at nominally normal incidence through the bottom of the microplate, can be used to illuminate a small region of the grating surface. A detection fiber for recording the reflected light is bundled with the illumination fiber. A series of illumination/detection heads are arranged in a linear fashion, so that reflection spectra are collected from a subset of wells within the same column of a 384-well microplate simultaneously. The whole plate is scanned by the illumination/detection heads so that each sensor can be addressed multiple times, and each column is addressed in sequence. The wavelengths of the reflected light are collected and used for analysis. A temperature-controlling unit minimizes temperature fluctuation.

In an alternative angular interrogation system, a polarized light, covering a range of incident angles, is used to directly illuminate the waveguide; light at specific angles is coupled into and propagate along the waveguide. The resonance angle at which a maximum in-coupling efficiency is achieved is a function of the local refractive index at or near the sensor surface. When target molecules in a sample bind to a cellular target in living cell system and subsequently triggers a cellular response within the bottom portion of the layer of the cell system or the biological systems, the resonance angle shifts. Such a system is described in, for example, U.S. Patent Publication No. US-2004-0263841, U.S. patent application Ser. No. 11/019,439, filed Dec. 21, 2004, and U.S. Patent Publication No. US-2005-0236554.

For cell-based assays of the present disclosure, live cells can be contacted with a compatibilized surface of a biosensor, for example, via culturing. The cell adhesion can be mediated through, e.g., three types of contacts: focal contacts, close contacts, and extracellular matrix (ECM) contacts. Each type of contact has its own characteristic separation distance from the surface. As a result, cell plasma membranes are about 10 to about 100 nm away from the substrate surface, so that optical biosensors of relatively short penetration depths are still able to sense the bottom portion of the cells proximate to the biosensor surface. A phenomenon that is common to many stimuli-induced cell responses is dynamic relocation or rearrangement of certain cellular contents; some of which can occur within the bottom portion of cells proximate to the biosensor surface. Dynamic relocation or rearrangement of cellular contents can include, for example, changes in adhesion degree, membrane ruffling, recruitment of intracellular proteins to activated receptors at or near a cell's surface, receptor endocytosis, and like phenomena. A change in cellular contents within the sensing volume leads to an alteration in local refractive index near the sensor surface, which manifests itself as an optical signal from the biosensor.

A significant characteristic of RWG biosensors for living cell sensing is the penetration depth or sensing volume. The RWG biosensor exploits an evanescent-wave that is generated by the resonant coupling of light into a waveguide via a diffraction grating. The guided light can be viewed as one or more mode(s) of light that all have a direction of propagation parallel with the waveguide due to the confinement by total internal reflection at the substrate-film and medium-film interfaces. Because the guided light mode has a transversal amplitude profile that covers all layers, the effective refractive index N of each mode is a weighted sum of the refractive indices of all layers:

N=f_{N}(n_{F},n_{S},n_{C},n_{ad},d_{F},d_{ad},λ,m,σ) (1)

where n_{F}, n_{S}, n_{C}, and n_{ad} are the respective refractive indices of the waveguide, the substrate, the cover medium, and the adlayer of the cells; d_{F} and d_{ad} are the respective effective thicknesses of the film, and the cell layer; λ is the vacuum wavelength of the light used; m=0, 1, 2, . . . is the mode number; and σ is the mode type number which equals to 1 for TE (transverse electric or s-polarized) and 0 for TM modes (transverse magnetic or p-polarized).

The guided light modes propagate parallel to the surface of a plane waveguide, to create an electromagnetic field (i.e., an evanescent wave) extending into low-refractive index mediums surrounding both sides of the film with a characteristic of exponential decay. The amplitude (Eₘ) of the evanescent wave decays exponentially with increasing distance z from the interface towards the cover medium or the substrate:

$${E}_{m} ( d ) = {E}_{m} ( 0 ) exp ( \frac{- z}{Δ {Z}_{J}} ) \qquad ( 2 )$$

with:

$$Δ {Z}_{J} = \frac{1 - σ}{{k ( {N}^{2} - {n}_{J}^{2} )}^{0.5}} + \frac{{σ [ {( N / {n}_{F} )}^{2} + {( N / {n}_{J} )}^{2} - 1 ]}^{- 1}}{{k ( {N}^{2} - {n}_{J}^{2} )}^{0.5}} \qquad ( 3 )$$

is the penetration depth of the evanescent tail of the waveguide mode that extends into the cover medium (J=c) or substrate (J=s). Based on the configuration of the biosensors used and the uniqueness of cell properties, the penetration depth of the TM₀ mode for Corning® Epic™ RWG biosensor microplates is, for example, about 150 nm. Such relatively short penetration depth or sensing volume is common to most types of label-free optical biosensor technologies including conventional SPR and RWG, so that the disclosure is applicable to other optical biosensor-based cell sensing.

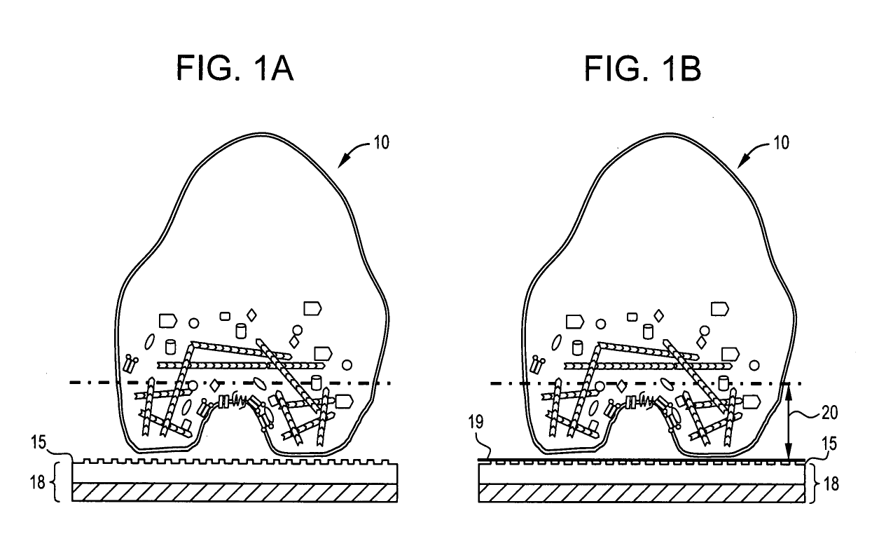

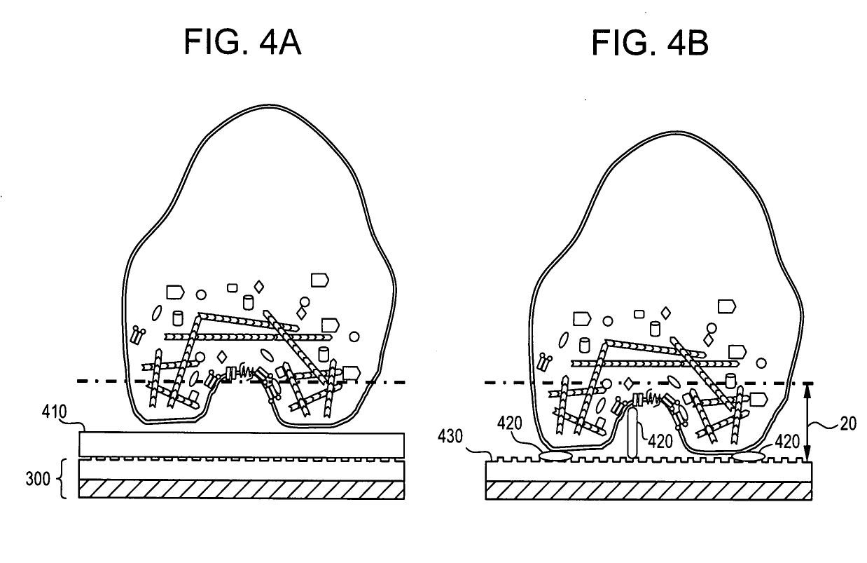

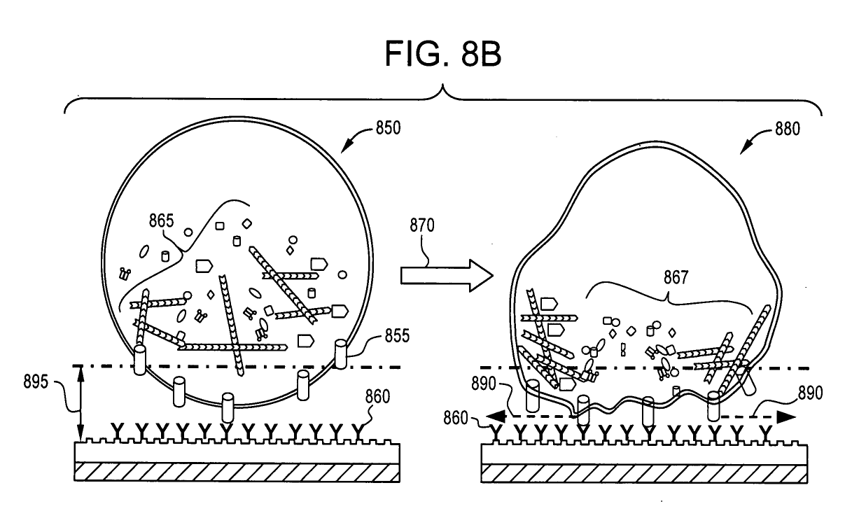

For whole cell sensing with RWG biosensor, the sensor configuration is considered as a non-classical three-layer system: a substrate, a waveguide film in which a grating structure is embedded, and a cell layer (e.g., as illustrated in FIG. 1A, FIG. 1B, FIG. 4A, FIG. 4B, and FIG. 8B), because of the large dimension of a living cell or a cell system.

A ligand-induced change in effective refractive index (i.e., the detected signal, or the optical signal) is, to first order, directly proportional to the change in refractive index of the bottom portion of cell layer:

ΔN=S(C)Δn_{c} (4)

where S(C) is the sensitivity to the cell layer, and Δn_{c} is the ligand-induced change in local refractive index of the cell layer sensed by the biosensor.

The Δn_{c} value is directly proportional to change in local concentrations of cellular targets or molecular assemblies within the sensing volume. This is because of a well-known physical property of cells—the refractive index of a given volume within cells is largely determined by the concentrations of bio-molecules, mainly proteins, which is the basis for the contrast in light microscopic images of cells.

The detected signal, or the optical signal, is a sum of mass redistribution occurring at distinct distances away from the sensor surface, each with unequal contribution to the overall response. This is because of the exponentially decaying nature of the evanescent wave. Taking the weighed factor exp(−zᵢ/ΔZ_{c}) into account, the detected signal occurring perpendicular to the sensor surface is governed by:

$$Δ N = S ( N ) α d {\sum }_{i} Δ {C}_{i} [ {e}^{\frac{- {z}_{i}}{Δ {Z}_{C}}} - {e}^{\frac{- {z}_{i + 1}}{Δ {Z}_{C}}} ] \qquad ( 5 )$$

where ΔZ_{c} is the penetration depth into the cell layer, α is the specific refraction increment (about 0.0018 for proteins), zᵢ is the distance where the mass redistribution occurs, and d is an imaginary thickness of a slice within the cell layer. Here the cell layer is divided into an equal-spaced slice in the vertical direction.

Theoretical analysis suggests that the detected signal, in terms of wavelength or angular shifts, is primarily sensitive to the vertical mass redistribution. Because of its dynamic nature, it is also referred to as a dynamic mass redistribution (DMR) signal. Beside the DMR signal, the biosensor is also capable of detecting horizontal (i.e., parallel to the sensor surface) redistribution of cellular contents. Theoretical analysis, based on the zigzag theory, shows that any changes in the shape of a resonant peak are mainly due to ligand-induced inhomogeneous redistribution of cellular contents parallel to the sensor surface (see Fang, Y., et al., (2006) “Resonant Waveguide Grating Biosensor for Live Cell Sensing,” Biophys. J, 91, 1925-1940). In addition, the DMR signal is a sum of all redistribution events within the sensing volume. This suggests that whole cell sensing with the biosensors of the disclosure is distinct from the aforementioned affinity-based assays, which directly measure the amount of analyte binding to the immobilized receptors.

### Mass Redistribution Cell Assay Technologies (MRCAT)

In commonly owned co-pending PCT Application No. PCT/US2006/013539, published Dec. 10, 2006, there is disclosed a non-invasive and manipulation-free cell assay methodology referred to as Mass Redistribution Cell Assay Technology (MRCAT). MRCAT uses an optical biosensor, particularly resonant waveguide grating (RWG) biosensor, to monitor the ligand-induced dynamic mass redistribution within the bottom thin portion of adherent cells. The DMR signal obtained represents an integrated cellular response, which resulted from the ligand-induced dynamic, directed, and directional redistribution of cellular targets or molecular assemblies. MRCAT permits the study of cell activities, such as signaling and its network interactions, and can also enable high throughput screening of ligand candidate compounds against endogenous receptors or over-expressed receptors in engineered cells or cell lines. The cell assays disclosed in the co-pending application were carried out using moderately adherent cells, including Chinese hamster ovary cells, human epidermoid carcinoma A431 cells, Cos7 cells, HeLa cells, primary cells, stem cells, and like cells. Cells were typically cultured directly onto unmodified surfaces of the RWG biosensors. These unmodified surfaces allowed self-attachment and growth of moderately adherent cells, and also enabled methods for the RWG biosensor to monitor ligand-induced cellular activities such as G protein coupled receptor (GPCR) signaling. However, these surfaces are not suitable for weakly adherent cells such as human embryonic kidney cells, or suspension cells such as Jurkat cells. Although weakly adherent cells can self-attach and grow on these unmodified surfaces, assays using optical biosensors, particularly RWG biosensors, failed because these weakly adherent cells tend to detach during washing or medium exchange (i.e., from cell culture medium to assay medium or buffer) or other external physical forces. The washing or medium exchange step is commonly involved in many types of cell-based assays. In contrast, suspension cells generally do not attach to these unmodified surfaces, and can typically retain their globular shape throughout cell culturing. Since optical biosensors, such as RWG biosensors and surface plasmon resonance (SPR), only permit the measurement of changes in local mass, mass density, or both, of cells near the sensor surface (e.g., within about 200 nm), it is difficult to detect any ligand-induced cell activities with suspension cells. Furthermore, the weak contact of a weakly adherent cell, or the no-contact of a suspension cell with the biosensor surface precludes any robust measurement of cellular activities induced by a ligand, because any external physical forces, such as the movement of the biosensor system or the fluidic movement in the sample solution addition step, could alter the distribution of cellular contents within the detection zone.

MRCAT starts with the interaction or contact of cells with the surface of a biosensor; typically, cells are cultured directly onto the surface of a RWG biosensor. Exogenous signals can mediate the activation of specific cell signaling, in many instances resulting in dynamic redistribution of cellular contents equivalent to dynamic mass redistribution (DMR). If signaling occurs within the sensing volume (i.e., the penetration depth of the evanescent wave) then the DMR can be manifested and monitored in real time by a RWG biosensor. Because of its ability for multi-parameter measurements, the biosensor has potential to provide high information content for cell sensing. These parameters include the angular shift (one of the most common outputs), the intensity, the peak-width-at-half-maximum (PWHM), the area, and the shape of the resonant peaks. The position-sensitive responses across an entire sensor can provide additional useful information regarding to the uniformity of cell states, for example, density and adhesion degree, as well as the homogeneity of cell responses for cells located at distinct locations across the entire sensor.

The DMR signals can yield valuable information regarding novel physiological responses of living cells. Because of the exponential decay of the evanescence wave tail penetrating into the cell layer, a target or complex of a certain mass contributes more to the overall response when the target or complex is closer to the sensor surface as compared to when it is further from the sensor surface. Furthermore, the relocation of a target or complex towards the sensor surface results in an increase in signal, whereas the relocation of a target or complex that moves away from the sensor surface leads to a decrease in signal. The DMR signals mediated through a particular target were found to depend on the cell status, such as degree of adhesion, and cell states, such as proliferating and quiescent states.

Because of the short sensing volume of commonly available optical biosensors such as RWG and SPR, the biosensor-based cell assays depend on close proximity of cells with the sensor surface. In addition, attachment of cells, growth of cells, or both, can be significant factors in the success of the present cell-based biosensor and its assay methods. In embodiments, the modified biosensor surfaces of the disclosure should be biocompatible with and support the attachment and growth of a wide variety of cell lines. In embodiments, the adherence of the cells to the modified biosensor surface can withstand manipulations such as washing and reagent dispensing.

Surfaces for Adherent Cells with Improved Assay Robustness

A typical RWG biosensor consists of a glass substrate, and a waveguide thin film in which a diffraction grating is embedded. The waveguide thin film has a high refractive index (n_{F}) material and a thickness of d_{F}, which is directly deposited onto a substrate of lower index (e.g., n_{S} is about 1.50 for glass). In embodiments such as for comparative purposes, live cells can be cultured directly onto the bare surface of the waveguide thin film, which supports attachment and growth of adherent cells, for example, transformed cell lines including Chinese hamster ovary (CHO) cells, A431 cells, HeLa cells, Cos7 cells, primary cells including human fibroblast cells, and like cells. Typical waveguide materials can include, for example, niobium oxide, tantalum oxide, titanium oxide, silicon nitride, or like materials, and combinations thereof.

In embodiments, the disclosure provides compatibilizer modified biosensor surfaces suitable for use with adherent cells and having improved assay robustness. In embodiments, a base layer or an extra thin layer of a compatibilizer, or compatibilizer and surface modifier, for example, having a thickness of about 1 to about 15 nanometers of a material, such as an inorganic material, can be deposited directly onto the biosensor surface of the waveguide as a thin film. The thickness of inorganic material coating can negatively impact the sensitivity of the RWG biosensor if the coating is too thick. A proper coating thickness in accord with the disclosure can be achieved by, for example, vapor deposition methods, soot gun methods, solution-based methods such as sol-gel formation or self-assembly, and like methods, or combinations thereof. The inorganic material can include, for example, a silicon oxide, tantalum oxide, titanium oxide, silicon nitride, or like materials, and combinations thereof. In embodiments, nanoparticles of the inorganic material can be directly deposited onto the waveguide thin film. In embodiments, the base layer or extra thin layer of compatibilizer or compatibilizer and surface modifier can be, e.g., discontinuous or incomplete, such as forming and having decoration resembling islands, patches, isolated particles, or like descriptions of the compatibilizer or compatibilizer and surface modifier combination.

Referring to the Figures, FIGS. 1A and 1B show cross-sectional configurations of two examples of RWG biosensors used for living cell sensing. In FIG. 1A a cell (10) is directly cultured onto the unmodified or bare surface of the waveguide thin film (15) of a RWG biosensor (18) having a waveguide grating atop a substrate. In FIG. 1B a cell (10) is cultured onto the surface of an inorganic thin film (19) which has been previously deposited onto the surface of the waveguide thin film (15). Here a thin film of silicon oxide of about 1 nm to about 5 nm is directly deposited onto the surface of a waveguide thin film of niobium oxide by vapor deposition method. Each biosensor associated cell has an approximate penetration depth (20).

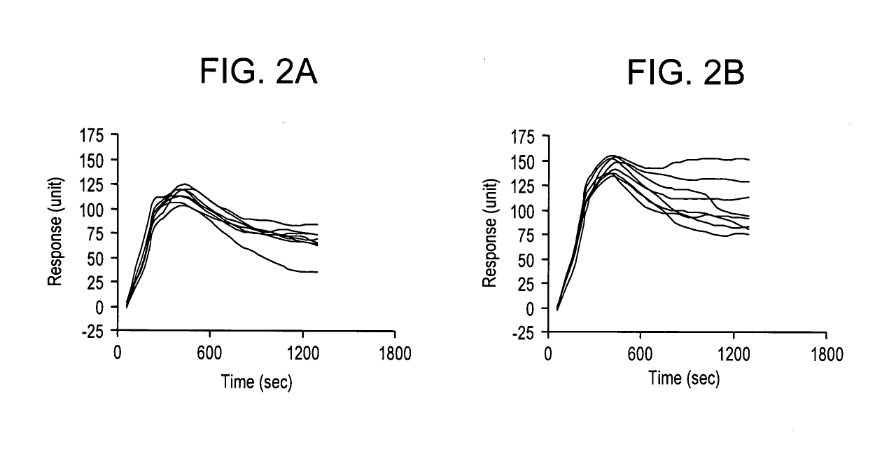

FIGS. 2A and 2B shows the results of adenosine triphosphate (ATP)-induced optical signals or DMR signals obtained from CHO-KCNQ cells on the respective configurations of the above mentioned FIGS. 1A and 1B. CHO-KCNQ is an engineered CHO cell having over-expressed a potassium ion channel KCNQ2. FIGS. 2A and 2B compare the ATP-induced DMR signals, response units versus time, of CHO-KCNQ cells: The FIG. 2A cells were cultured on an unmodified or bare niobia surface of the waveguide thin film of a Corning® Epic™ RWG biosensor; and FIG. 2B cells were cultured on a biosensor having, for example, a discontinuous thin film modified waveguide surface of silicon dioxide particles. The FIG. 2A cells produced an average signal of 115 pm, having a standard deviation of 7 pm, and a Coefficient of Variability (CV) (a measure of assay reproducibility), of 6%, whereas the FIG. 2B cells produced an average signal of 144 pm, having a standard deviation of 8 pm, and a CV of 6%. Thus, methods of the disclosure provide assays having high reproducibility or low variability, for example, having a coefficient of variability (CV) of less than or equal to about 10%, less than or equal to about 8, less than or equal to about 6%, less than or equal to about 4%, and like CVs.

### Surfaces for Weakly Adherent Cells

In embodiments, the disclosure provides RWG biosensors having surfaces that can promote cell attachment and growth, and also optionally enable robust cell assays for weakly adherent cells. In embodiments, the surface of a waveguide thin film is modified with a cell anchoring material known to promote cell attachment; the cell anchoring material can form a series of clusters or islands that randomly distribute on the sensor surface. The cell anchoring materials that are known to promote cell attachment include, for example, a biological material, a polymeric material, or an inorganic material. In embodiments, the cell anchoring material can be referred to as a compatibilizer, a surface modifier, or the combination of both, in accordance with the abovementioned definitions.

In embodiments, the cell anchoring material can be a biological material. The biological material includes, for example, cell surface receptor-interacting molecules, cell adhesion polypeptides, cell adhesion peptide, or like material. The cell adhesion polypeptides include, for example, gelatin, fibronectin, laminin, fibronectin proteolytic fragment, collagen, poly-D-lysine, or like materials, and combinations thereof. The cell adhesion peptides include, for example, Arg-Gly-Asp (RGD), an RGD containing peptide, or like materials. The RGD resides in the cell attachment region of fibronectin and has been intensively studied as a cell-binding sequence. The cell surface receptor-interacting molecules can include, for example, polyclonal antibodies, monoclonal antibodies, single chain antibodies (scFv), F(ab) fragments, F(ab′)₂ fragments, Fv fragments, peptides, proteins, and like material, which can specifically bind to the cell surface receptor such as integrin or integrin receptor, immune receptor, G protein-coupled receptor (GPCR), glycoprotein, or like material.

In embodiments, the cell anchoring material can be a polymeric material. The polymeric material can include, for example, a naturally occurring or synthetic polysaccharide. In embodiments, the cell anchoring material can be an inorganic material. The inorganic material can include, for example, silica, silicate, like silicon oxides and derivatives thereof such as hydrophobic Aerosils™, or like materials. In embodiments the cell anchoring materials can be applied to a biosensor surface to render it more receptive or interactive, through either direct or indirect mechanisms, with a subsequently contacted or cultured cell so as to enhance immobilization of the cell to the biosensor. In embodiments the cell anchoring material can be combinations of one or more of a biological material, a polymeric material, or an inorganic material.

In embodiments, islands of cell anchoring material can be patterned onto the surface of a waveguide thin film so that each island has a dimension that is much smaller than the size of an adherent cell, for example, around about 10 micrometers in thickness, area coverage diameter, or both. For example, the cell anchoring material islands could be square, circular, irregular, or like geometries, and combinations thereof. The diameter or width of a cell anchoring material island can be, for example, 100 nm, 500 nm, 1,000 nm, 2,000 nm, and like dimensions including intermediate dimensions and intermediate ranges. In embodiments, the density of the cell anchoring material islands can be high enough such that there is at least one island per cell and the island contacts the cell surface area, such as a cell invagination area, and other than the area directly contacted with the waveguide surface, when the cells are attached to the surface. Micro-patterned or nano-patterned methods, such as AFM (atomic force microscopy) cantilever-based deposition, nano-printing, or like methods, and combinations thereof can also be used to form or distribute the islands on the surface.

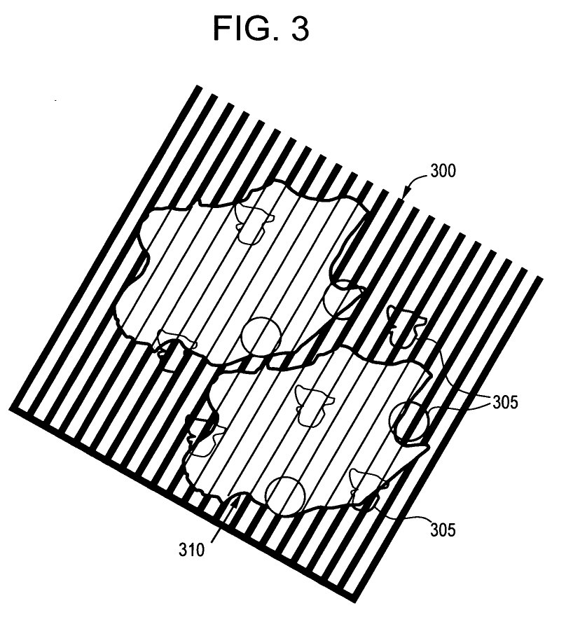

FIG. 3 is a schematic that illustrates clusters or islands of a biological material on the surface of a RWG biosensor can be anchor points for cell attachment and growth. FIG. 3 shows a low-density biological material (305) coating on a resonant waveguide grating biosensor (300). Typically, biological materials in embodiments of the disclosure can form small clusters which are randomly distributed about the surface of the biosensor substrate. These clusters or islands of biological material can serve as anchoring points that can permit cell attachment and prompt appropriate cell growth, and enable a robust cell assay.

FIGS. 4A and 4B show two example configurations of surface modified RWG biosensors used for living cell sensing and which biosensors have differential sensitivities to stimulus-induced change in local mass or mass density. FIG. 4 provides a schematic that illustrates different sensitivities of RWG biosensors to stimulus-induced change in local mass or mass density (e.g., mass redistribution) of living cells attached to two different surfaces. FIG. 4A has a layer or film of biological material (410) coated on the top or grating surface of a biosensor (300) so that the cells are away from the sensor surface. Because of the limited detection zone or sensing volume of the sensor, the biosensor is expected to be relatively insensitive to any responses of the cells induced by stimulation. FIG. 4B in contrast has a low-density non-film or discontinuous biological material (420) coated on the biosensor surface (300), so that the cells are much closer to the biosensor surface compared to the cells of FIG. 4A, which can lead to the higher sensitivity to any DMR responses in cells (compare separation of respective broken phantom lines from the grating surface (300)). Here two types, such as by compositional difference, orientational difference, or both of biological material islands (420) are presented. The islands or clusters of biological materials can act as anchoring points to hold the cells to the biosensor contact surface (430), but through distinct mechanisms. The first one promotes the formation of cell adhesion complexes, whereas the second one interacts with the invagination area of the cell surface, and thus provides extra force to enhance the cell attachment.

### Low Density Gelatin Surface Coating

One method that can be used to prepare low-density gelatin surfaces comprises, for example, dispensing highly dilute aqueous gelatin solutions, e.g., 0.000005-0.0025% by weight concentration, into wells of a microtiter plate, such as 96-well microplates, 384-well microplates, or 1,536-well microplates. The solutions are then dried by slow evaporation in a desiccator at room temperature and low room humidity, such as 20% relative humidity, for a given period of time such as 1-10 days, until it is completely dried to produce a low density gelatin decorated surface.

### Analysis of Low-Density Gelatin Coated Surfaces

Atomic Force Microscope (AFM) and Scanning Electron Microscope (SEM) images were obtained for low-density gelatin surfaces prepared by slow evaporation of 0.0015% solutions from a glass slide with virtual wells (see, e.g., U.S. Pat. No. 6,908,760; K. A. Thompson, “Virtual Wells: Combinatorial Biology Demands Ultra-High-Throughput Screening”, http://www.netsci.org/Science/Screening/feature04.html, Jan. 8, 1998, pp. 1-6.). The SEM images unexpectedly showed that the gelatin coatings were not thin continuous films, but were instead nanoparticulates. The nanoparticles had a cubic structure with dimensions of about 80 nm on each side. The surface coverage was less than about 1%. The contact angles of clean uncoated glass and the gelatin-coated glass were measured and found to be markedly different. The gelatin-coated glass was significantly more hydrophilic and more water-wettable than the uncoated glass as summarized in the accompanying table below.

| Surface | Water Contact Angle (deg) |
|---|---|
| Uncoated glass | 78 |
| Gelatin-coated glass | 39 |

The SEM (5,000×) and AFM (20,000×) the measurements are summarized in the accompanying table below.

| Metric/Magnification | 5,000X | 20,000X |
|---|---|---|
| Area Measured (μm²) | 2.70 | 0.17 |
| Number of Particles Counted | 445 | 31 |
| Particle Density (particles/μm²) | 165 | 182 |
| Surface Coverage (%) | about 0.76 | about 0.78 |
| Ave. Particle Dimension (nm) | — | 78 |

### Gelatin-Coated Surface Provides Superior Adherence for HEK293 Cells

When HEK293 cells were cultured on uncoated Corning's Epic™ sensor surfaces, these surface supported the attachment and growth of HEK293 cells. However, these cells were more easily displaced upon washing with, for example, serum-free media, buffer, or both, than cells on low-density gelatin-coated surfaces.

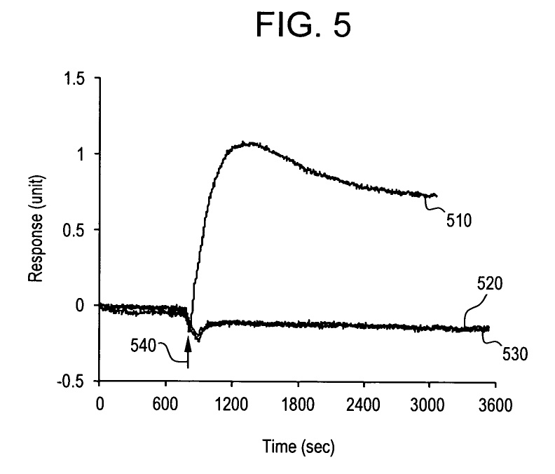

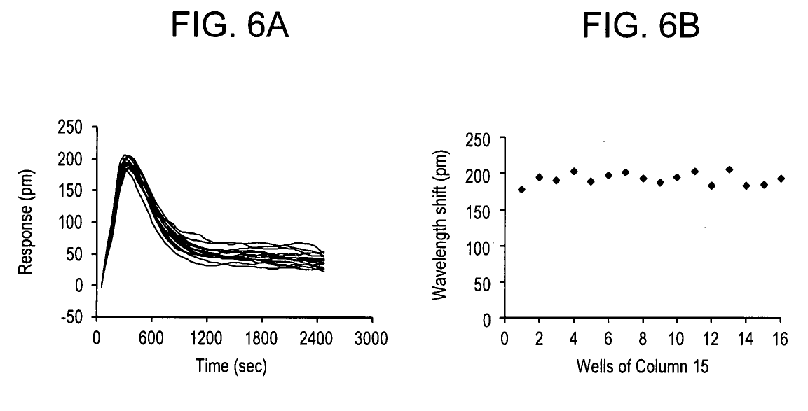

In contrast, the attachment and biosensor-based cell assays with gelatin coated sensor surfaces of the disclosure exhibited a clear dependence on the gelatin coating density. Three surface densities were individually prepared by slow evaporation of 0.0015%, 0.1% and 1.5 weight % gelatin solutions, as described above, covering the biosensor surfaces. The resultant respective gelatin surfaces were referred to as low-, medium-, and high-density gelatin surfaces, respectively. HEK293 cells were separately cultured onto these surfaces. The results showed that while all three of these gelatin-coated surfaces support the attachment and growth of HEK293 cells, only the low-density gelatin surfaces supported the strong adherence of these cells, and enabled robust biosensor-based cell assays (FIG. 5 and FIG. 6). Light microscopic images showed the HEK293 cells can attach to and grow at an appropriate rate (i.e., the cell doubling rate of the HEK cell numbers during culture was found to be around 16 hours, comparable to growth rates observed for these cells cultured in standard TCT (tissue culture treated plate)) on these low-density gelatin-coated surfaces, and these cultured cells remained almost completely intact after washing the cultured cells twice with the 1×HBSS buffer (1× Hank's balanced salt solution, 20 mM Hepes, ph7.0) (data not shown). Furthermore, the microscopic images indicated that the low-density gelatin-coated surfaces supported the attachment and growth of HEK293 cells, regardless of the gelatin type (such as from different vendors including Sigma, BD Biosciences, or others), molecular weight, and bloom number.

The biosensor-based cell assays, using adenosine triphosphate (ATP), as an inducer (an agonist of endogenous P2Y receptors in HEK 293 cells), showed that only the cultured cells on these low-density gelatin surfaces led to robust cell assays. P2Y receptors belong to the family of G protein-coupled receptors. HEK 293 cells endogenously express several isoforms of P2Y receptors. FIG. 5 shows a comparison of ATP-induced optical signals obtained from HEK293 cells that were cultured onto three different gelatin-density coated surfaces: low-density (510), medium-density (520), and high-density (530) gelatin-coated surfaces. Only the low-density gelatin-coated surface enabled the detection of the ATP-induced DMR signals of HEK293 cells. The arrow (540) indicates when the stimulus, an ATP solution, was introduced.

FIGS. 6A and 6B show the reproducibility and robustness of a cell assay of the disclosure using HEK293 cells that were cultured on a low-density gelatin-coated 384-well Epic™ biosensor microplate. A 10 micromolar ATP solution was added to selected well of column 15 of the microplate. The ATP-induced DMR response signals within one column (16 wells) were recorded in real time in seconds and plotted in FIG. 6A. The amplitudes of the initial positive DMR (P-DMR) event (wavelength shift) of the ATP-induced DMR signals were plotted as a function of well numbers 1-16 in FIG. 6B. The assay Coefficient of Variability (CV), or reproducibility, was about 4%, indicating a relatively high reproducibility, with an average signal of 193 picometers (pm), and a standard deviation of 8 pm.

### GRGDS-Modified Surface Enables Strong Adherence for HEK293 Cells

Synthetic peptides containing the arginine-glycine-aspartate (RGD) sequence motif are active modulators of cell adhesion. This tripeptide motif can be found in proteins of the extracellular matrix. Integrins link the intracellular cytoskeleton of cells with the extracellular matrix by recognizing this RGD motif. The covalent attachment of RGD peptides to biosensor modified surfaces is a useful alternative to control cell adhesion to biomaterials of interest. Many active RGD and like peptides are commercially available.

To covalently couple RGD-like peptides to the biosensor surface, the biosensor was coated sequentially with a thin layer of silicon oxide (about 3 nm), aminopropylsilane (about 1-3 nm), poly(ethylene-alt-maleic anhydride) (EMA) (forming polymeric clusters), and finally a RGD-like peptide (Gly-Arg-Gly-Asp-Ser, GRGDS). Specifically, a thin layer of silicon oxide was deposited onto the Corning Epic™ niobium oxide waveguide surface up to 3 nm. Afterwards, the biosensor was coated with an aminopropylsilane solution of about 0.1 vol/vol %, washed and dried, followed by the interaction with an EMA solution of about 0.01 wt/vol %, and washed and dried. Finally, a GRGDS solution was applied to the EMA biosensor surface. After the coupling reaction, the surface was washed and dried. The different GRGDS densities were achieved by immobilizing a same volume (about 100 microliters) of solution of GRGDS at different concentrations. Corning Epic™ 96-well biosensor microplates were used. Here, the formation of islands or clusters of GRGDS are achieved by using an amino-reactive polymer (e.g., EMA) and by controlling the concentration of GRGDS in the final coupling step.

The resultant GRGDS-modified plates were tested for the growth of the HEK293 cells and for the cell assays. Results showed that the HEK293 cells on the GRGDS-modified surfaces grew significantly faster than those on the uncoated surfaces. The cultured HEK cells on the GRGDS-presenting surfaces can survive through the washing or medium exchange procedure, as shown by light microscopy imaging. Using ATP as a stimulation agent, the biosensor-based cell assays were examined for these HEK cells cultured on the GRGDS presenting surfaces. The RGD surface coupling was achieved through the N-terminal amine of the tripeptide, pentapeptide, or like materials, reacting with the carboxy group of the EMA.

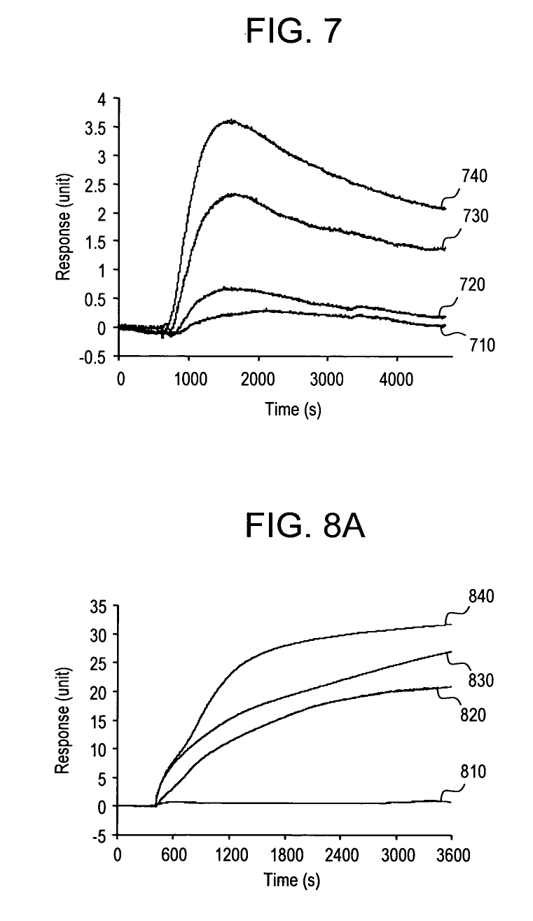

FIG. 7 shows an example of a standard activity assay plot of ATP-induced DMR signals for HEK293 cells cultured on separate compatibilized RWG surfaces and having different concentrations of a surface-bound or surface-conditioned tripeptide compatibilizer. ATP (20 micromolar) was used to induce DMR signals of HEK293 cells that were cultured on the GRGDS-derivatized EMA RWG surfaces. Four different concentrations (10, 30, 100, and 250 micromolar; reference numerals (710), (720), (730), and (740), respectively) of the GRGDS peptide were used in separate surface derivatization treatments. Results showed that an increasing response intensity induced by ATP for the cultured HEK cells was found to correlate well with increasing the GRGDS compatibilizer concentration.

### Surfaces for Suspension Cells

In embodiments, the disclosure provides compatibilized surfaces and methods for making that enable robust interaction of suspension cells with the compatibilized RWG surface, and enable probe ligand-induced activity in suspension cells. In embodiments, the compatibilized surface presents a reactive specie, such as amine-reactive functional groups (e.g., N-oxysuccinimide esters, N-hydroxysuccinimide (NHS), and N-hydroxysulfosuccinimide (Sulfo-NHS)), or thiol-reactive functional groups (e.g., methanethiosulfonate). The cell surface amine-presenting molecules (e.g., proteins, lipids) of suspension cells, such as Jurkat cells, can interact with these reactive functional groups, and thus form covalent bonds. The suspension cells therefore become, for example, covalently associated with the compatibilized surface. However, the suspension cells generally maintain their globular shape as presented in solution.

In embodiments, the compatibilized RWG surface provides or present bio-interacting molecules having functional groups, where the compatibilizer surface molecules can recognize and interact with certain of a cell's surface molecules, such as an antigen, a receptor, a lipid, and like cell components or cell surface molecules, thus enabling the attachment of suspension cells to the bio-sensor surface. The bio-interacting molecules of the compatibilizer can include, for example, an antibody, or like ligand or molecular entity, which can specifically bind to a cell's surface molecule or an engineered cell surface molecule. In embodiments, micropatterning or nanopatterning methods can be used to deposit materials having functional or reactive groups onto the compatibilized surface of a RWG biosensor to form micro- or nano-domains of these materials, such that there is at least one domain per cell when the cells are attached.

In embodiments, the disclosure also provides a method to assay ligand-induced cellular activity in suspension cells using a compatibilized optical biosensor. Such an approach was heretofore believed to be unworkable since suspension cells are generally not well adhered onto the surface of conventional substrates.

Jurkat cells are an immortalized line of T lymphocyte cells that are used to study, for example, acute T cell leukemia and T cell signaling. Jurkat cells are also useful for their ability to produce interleukin 2. Their primary use, however, is to determine the mechanism of differential susceptibility of cancers to drugs and radiation. The Jurkat cell line was established in the late 1970s from the peripheral blood of a 14 year old boy with T cell leukemia. Different derivatives of the Jurkat cell line can now be obtained from cell culture banks (e.g., ATCC 2) that have been mutated to lack certain genes. The Jurkat cells are a suspension type of cells, wherein the cell culture is carried out in the medium, and the cells do not contact with and grow on the surface of a substrate.

The Jurkat cells, which present antigens at the cell's surface, were contacted with four different surface types: a bare waveguide substrate having a thin layer of silicon oxide (i.e., SiOx/Nb₂O₅, uncoated biosensor substrate) (810), a SiOx/Nb₂O₅ surface having anti-human CD3 (anti-CD3) (820), a SiOx/Nb₂O₅ surface having amino-reactive polymer EMA coating (EMA) (830), and a SiOx/Nb₂O₅ surface having covalently coupled anti-human CD3 (i.e., anti-CD3-EMA) (840). The anti-CD3 surface was achieved by incubating a solution containing anti-human CD3 antibody (25 μg/ml) onto the bare SiOx/Nb₂O₅ surface for 1 hour, followed by washing and drying. The EMA surface was prepared as described above, i.e., by sequentially coating the bare SiOx/Nb₂O₅ surface with aminopropylsilane and then EMA. The anti-CD3/EMA surface was prepared by incubating anti-human CD3 (25 μg/ml) solution with the EMA surface for 1 hour, followed by blocking with ethanolamine at 1% for 1 hour. The Jurkat cells were incubated on either surface for about 2 hours before assays. The kinetic response, before and after the pseudo-RACK1 addition, was recorded in real time.

FIG. 8A shows a plot of optical signals obtained from Jurkat cells when mediated by a stimulus such as a cell permeable peptide (pseudo-RACK1) on various biosensors having different surface preparations and properties. Pseudo-RACK1 is a peptide (OH-lys-lys-trp-lys-met-arg-arg-asn-gln-phe-trp-ile-lys-ile-gln-arg-cys----cys-ser-val-glu-ile-trp-asp-OH) and activates intracellular protein kinase C (PKC). Results showed that on bare SiOx/Nb₂O₅ the peptide pseudo-RACK1 did not led to any detectable optical signal, while on the other three types of surfaces, significant optical signals were obtained following the stimulation with pseudo-RACK1 at 9 μM. Interestingly, the dynamic profiles were similar to the Jurkat cells on these three surfaces, although the amplitude and kinetics exhibited some differences.

FIG. 8B shows a schematic representation of one scenario of Jurkat cell stimulation and sensing using the surface modified biosensors and methods of the disclosure. A Jurkat cell (850) having surface antigen CD3 (855) was contacted with a compatibilized anti-CD3-presenting optical biosensor surface (860). However, the contacted cells appeared to maintain their general globular shape before stimulation, as determined by light microscopy. After a chemical stimulation event (870) with, for example, pseudo-RACK1, the cell (850) undergoes morphological changes or deformations including the cell membrane (880), changes in other intracellular targets (865), and like changes, due to, for example, the activation of PKC induced by the pseudo-RACK1. Such morphological changes can drive other like cell surface antigens (855) to interact with the anti-CD3-presenting (860) sensor surface, and perhaps can drive other intracellular components (867) to congregate or assemble in the sensing volume or penetration depth region (895) of the biosensor. Through by perhaps a “rolling” motion or movement, the cell (880) becomes flatter compared to the uninduced cell (850) and increases the cell's contact area with the biosensor surface, as confirmed by light microscopy imaging (data not shown). Cell “rolling” can be roughly be analogized, for example, to a lock-and-key process for enzyme activity where for example, each cell contact with the biosensor surface can lead to or promote a cascade of additional or further cell contacts with the biosensor surface. The broken arrows (890) schematically indicate the possible directionality of the rolling motion of the cell as a result of the surface antigen interaction and subsequent stimulation. This and like phenomena associated with directional mass redistribution events are readily detected and analyzed in embodiments of the disclosure (see for example Fang, et al., ref 1 below at page 47)

It is known that fibronectin promotes the attachment of suspended cells to collagen and promotes the attachment of suspended cells directly to a tissue culture substrate. For example, U.S. Pat. No. 4,517,686, mentions a 108 amino acid polypeptide or its biologically active fragments which have the cell-attaching activity of fibronectin, and which polypeptides can be used to prepare substrates which promote the attachment of cells thereto. See also Li, et al., Biomolecules, 2006, 7, 1112-1123, “Investigation of MC3T3-E1 Cell Behavior on the Surfaces of GRGDS-Coupled Chitosan.”

### Applicability to Other Optical Biosensors

The disclosure also provides methods to modify other optical biosensors, such as SPR, as well as other biosensor, such as an electric impedance-based biosensor, so that cells can attach and grow on these surfaces, and can also permit the attached cells to be assayed. Specifically, SPR uses a thin layer of gold film as a substrate. The gold surface can be modified using similar protocols as mentioned above. Low-density coating of biological material or nanopatterned biological material can also be prepared (see for example U.S. Pat. No. 6,893,705). Cells cultured onto these surfaces can be used for assaying ligand-induced cellular activities of both adherent and weakly adherent cells. In embodiments, biosensor surfaces having, for example, reactive species or bio-interacting molecules, can also be made on the gold substrate. These modified biosensor surfaces can also be applied to assay ligand-induced cellular activities of suspension cells.

The disclosure has been described with reference to various specific embodiments and techniques. However, it should be understood that many variations and modifications are possible while remaining within the spirit and scope of the disclosure.

What is claimed is:

1. An apparatus for measuring ligand-induced cell activity, the apparatus comprising:
an optical biosensor having a contact surface comprising:
a compatibilizer zone having a compatibilizer directly or indirectly connected to the surface of the biosensor; and
a cell zone having at least one cell associated with at least one compatibilizer,
the compatibilizer comprises at least one of:
an isolated compatibilizer,
an island of two or more compatibilizers,
a discontinuous film comprised of a plurality of compatibilizers,
or combinations thereof.

2. The apparatus of claim 1 wherein the cell comprises at least one of a surface adherent cell, a weakly adherent cell, a suspension cell, or combinations thereof, the compatibilizer zone comprises the region occupied by a compatibilizer and situated between the biosensor's surface and a cell, the compatibilizer comprises: a gelatin, a peptide, an antibody, a metal oxide, a mixed metal oxide, or combinations thereof on the biosensor at an as-applied concentration of from about 1 to about 1,000 micromolar.

3. The apparatus of claim 2 wherein the compatibilizer comprises a gelatin, a metal oxide, a mixed metal oxide, a peptide, or mixtures thereof as nanoparticulates having a surface coverage on the biosensor of from about 0.01% to about 10%.

4. The apparatus of claim 2 wherein the compatibilizer zone further comprises a surface modifier zone which comprises the region occupied by a surface modifier situated between the biosensor's surface and the compatibilizer, the surface modifier comprises at least one nanoparticulate of: a metal oxide, a mixed metal oxide, a surface treated metal oxide, a surface treated mixed metal oxide, a gelatin, or combinations thereof, and having a particle diameter of from about 1 to about 15 nanometers, and the surface modifier has a surface area coverage on the biosensor of from about 0.01% to about 10% of the total biosensor surface.

5. A method of making the apparatus of claim 1, the method comprising:
decorating a surface of the biosensor with a compatibilizer to form a compatibilized biosensor contact surface; and
attaching a live cell to the compatibilizer-decorated biosensor surface, the decorating results in a biosensor surface having from about 10 to about 95 percent compatibilizer coverage based on the available biosensor contact surface area, and attaching the live cell to the compatibilizer decorated biosensor surface results in a sensing surface having from about 10 to about 100 percent of available compatibilizer covered surface or sites covered by associated cells.

6. The method of claim 5 wherein the decorating comprises contacting the biosensor surface with a solution or suspension containing at least one compatibilizer, the attaching comprises contacting the compatibilizer-decorated biosensor surface with a cell.

7. The method of claim 6 wherein the decorating further comprises contacting the biosensor surface with a surface modifier prior to contacting the biosensor surface with a compatibilizer.

8. The method of claim 5 further comprising treating the compatibilized biosensor surface with a blocking agent prior to contacting the compatibilized biosensor surface with a cell suspension.

9. A method of measuring ligand-induced cell activity, the method comprising:
contacting an optical biosensor of claim 1 with a ligand candidate; and
measuring the cell's optical response to the ligand contact with a detection system, wherein the ligand candidate comprises at least one of:
a drug candidate small molecule,
a drug candidate biologic molecule,
a drug candidate small molecule-biologic conjugate,
a bacterium,
a virus,
or combinations thereof, and wherein the ligand candidate has no affinity with, or low affinity for:
an uncoated biosensor surface;
a surface modifier treated biosensor surface;
a compatibilizer treated biosensor surface; or
a compatibilizer and surface modifier treated biosensor surface, measuring the cell's optical response to the ligand contact comprises detecting and determining the difference between the refractive index of the incident and reflected light, and correlating the DMR signals to the cell's activity.

10. A method to assay ligand-induced cell activity, the method comprising:
incubating a medium having at least one cell therein with a contact surface of an optical biosensor until a cell attaches to the biosensor surface, the biosensor surface having a compatibilizer attached-to but incompletely covering the biosensor surface, the compatibilizer having a functional group that can interact with a cell surface molecule;
contacting the biosensor having an attached cell with a ligand candidate; and
monitoring the cell response to the ligand contact with a detection system.
